(19) United States
(12) Patent Application Publication
(10) Pub. No.: US 2004/0215229 A1
(43) Pub. Date: Oct. 28, 2004

(54) Stent delivery system and method

(75) Inventor: James Coyle, Somerville, MA (US)

Correspondence Address: Mr. Janis Biksa, Patent Counsel, Intellectual Properties, Medtronic AVE, 3576 Unocal Place, Santa Rosa, CA, 95403, US

(73) Assignee: Medtronic AVE, Inc.

(21) Appl. No.: 10/421,290

(22) Filed: Apr. 22, 2003

(51) Int. Cl. A61F 2/02; A61M 29/00

(52) U.S. Cl. 606/200; 623/1.11

(57) ABSTRACT

A filter is deployed adjacent an occlusion in a vessel. A device including a stent and a sheath is advanced to the occlusion, the sheath is retracted to deploy the stent, and the stent is post dilated by a balloon of the device. The sheath is advanced through the deployed stent and over the balloon and over the filter. Since the balloon and the filter are enclosed within the sheath, any possibility of the balloon and/or the filter catching on the stent during retraction is eliminated.

### BACKGROUND OF THE INVENTION

1. Field of the Invention

The present invention relates to an intra-vascular device and method. More particularly, the present invention relates to a delivery system for deploying endoluminal prostheses within the lumens of the body and to a method of using the same.

2. Description of the Related Art

Human blood vessels often become occluded or completely blocked by plaque, thrombi, other deposits, emboli or other substances, which reduce the blood carrying capacity of the vessel. Should the blockage occur at a critical place in the circulatory system, serious and permanent injury, or even death, can occur. To prevent this, some form of medical intervention is usually performed when significant occlusion is detected.

Various types of intervention techniques have been developed which facilitate the reduction or removal of the blockage in the blood vessel, allowing increased blood flow through the vessel. One technique for treating stenosis or occlusion of a blood vessel is the deployment of a stent. However, stent deployment inherently carries the risk of embolism caused by the dislodgement of the blocking material, which then moves downstream.

To capture the dislodged blocking material, typically, a filter device is initially deployed downstream of the stenosis. After deployment of the filter device, a stent delivery system is then advanced over a wire of the filter device and the stent is deployed.

The stent delivery system is then removed and a post dilation balloon assembly is again advanced over the wire of the filter device. A balloon of the post dilation balloon assembly is inflated to dilate the stent. The post dilation balloon assembly is then removed and a filter retrieval device is again advanced over the wire of the filter device and retrieves the filter.

As should be readily apparent, inserting and removing the various systems and devices is relatively complex, time-consuming, and carries an increased risk of infection.

### SUMMARY OF THE INVENTION

In accordance with an embodiment of the present invention, a method includes deploying a protective device comprising a protective element, e.g., a filter, adjacent an occlusion in a vessel. The method further includes advancing a device comprising a stent and a sheath over a guide wire of the protective device, retracting the sheath to deploy the stent and post dilating the stent by inflating a stent dilation balloon. The sheath is advanced through the stent and over the stent dilation balloon and over the protective element. The stent dilation balloon and the protective element enclosed within the sheath are retracted.

Since the stent dilation balloon and the protective element are enclosed within the sheath, any possibility of the stent dilation balloon and/or the protective element catching on the stent during retraction is eliminated. In this manner, dislocation of the stent and the associated complications are avoided.

Further, since only a single device is used to deliver the stent, post dilate the stent, and recover the protective element in accordance with one embodiment, the entire procedure is relatively simple, quick, and has a minimal associated risk of infection.

Also in accordance with one embodiment of the present invention, a device for delivering a stent includes: an inner member defining a guide wire lumen; a tip mounted on the inner member; a stent dilation balloon mounted over the inner member, wherein the stent is mounted over the stent dilation balloon; an outer shaft coupled to the stent dilation balloon, wherein a balloon dilation lumen is defined by an annular space between the inner member and the outer shaft; a sheath constraining the stent; and a sheath movement mechanism for advancing the sheath past the tip.

### BRIEF DESCRIPTION OF THE DRAWINGS

FIG. 1 is a side view, in partial cross-section, of a protective device deployed within a parent artery or vessel of a patient adjacent to an occlusion;

FIG. 2 is a side view, in partial cross-section, of a stent delivery, dilation and filter recovery device within the vessel;

FIG. 3 is a side view, in partial cross-section, of the stent delivery, dilation and filter recovery device of FIG. 2 within the vessel after deployment of a stent;

FIG. 4 is a side view, in partial cross-section, of the post deployment dilation of the stent;

FIG. 5 is a side view, in partial cross-section, of the enclosure of a stent dilation balloon and a tip within a sheath;

FIG. 6 is a side view, in partial cross-section, of the enclosure of a protective element within the sheath;

FIG. 7 is a side view, in partial cross-section, of a stent delivery, dilation and filter recovery device;

FIG. 8 is enlarged view of a region VIII of FIG. 7; and

FIG. 9 is enlarged view of a region IX of FIG. 7.

Common reference numerals are used throughout the drawings and detailed description to indicate like elements.

### DETAILED DESCRIPTION

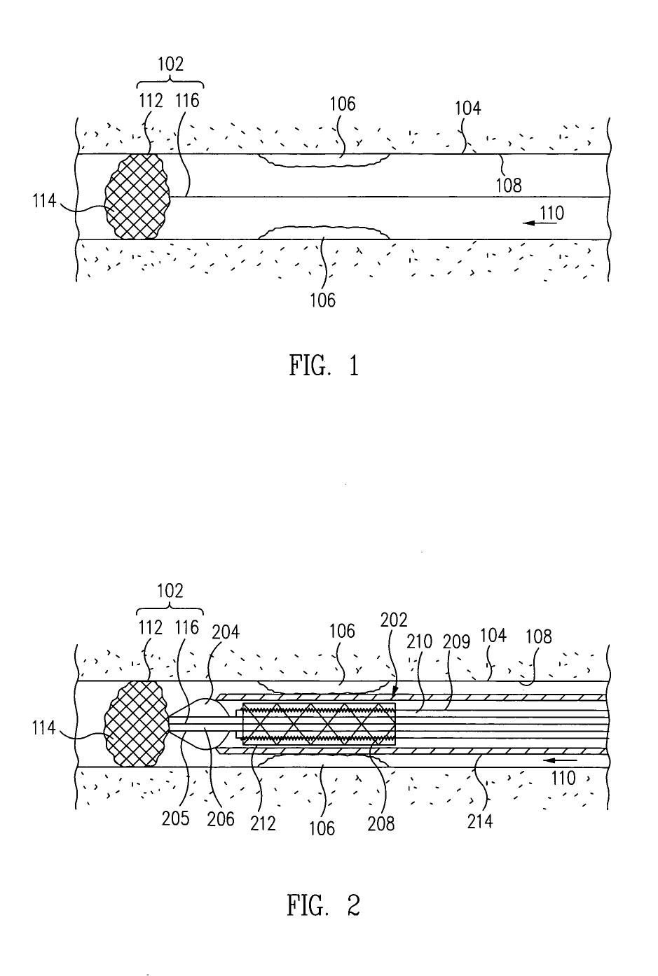

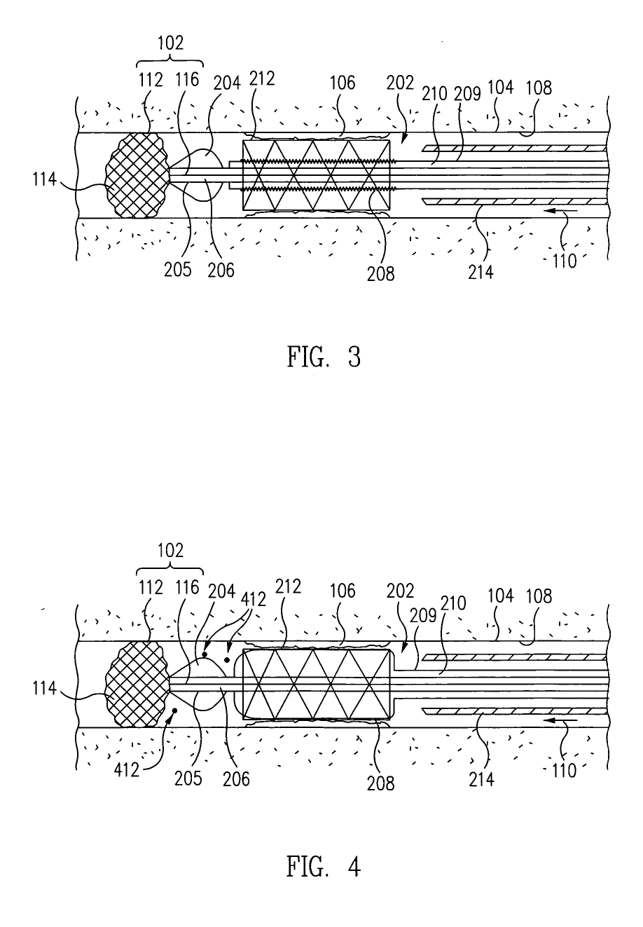

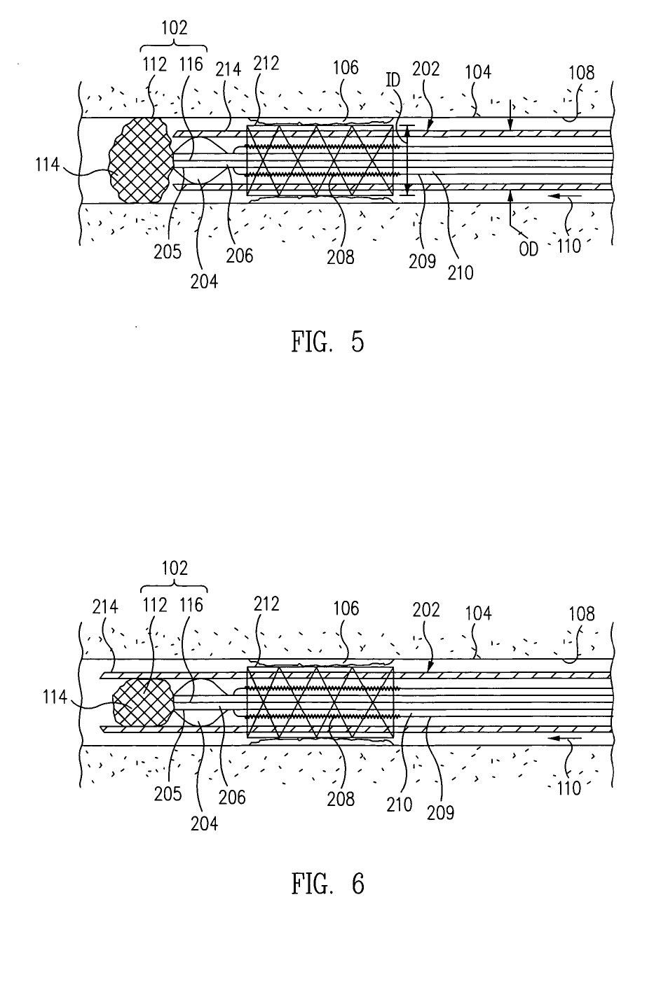

In an embodiment according to the present invention, a method includes deploying a protective device 102 (FIG. 1) comprising a protective element 112, e.g., a filter, adjacent an occlusion 106 in a vessel 104. The method further includes advancing a device 202 (FIG. 2) comprising a stent 212 and a sheath 214 over a guide wire 116 of protective device 102, retracting sheath 214 (FIG. 3) to deploy stent 212 and post dilate stent 212 (FIG. 4) by inflating a stent dilation balloon 208. Sheath 214 is advanced through stent 212 and over stent dilation balloon 208 (FIG. 5) and over protective element 112 (FIG. 6). Stent dilation balloon 208 and protective element 112 once enclosed within sheath 214 are retracted.

Since stent dilation balloon 208 and protective element 112 are enclosed within sheath 214, any possibility of stent dilation balloon 208 and/or protective element 112 catching on stent 212 during retraction is eliminated. In this manner, dislocation of stent 212 and the associated complications (repair or the need for repeated retraction attempts) are avoided.

Further, since only a single device 202 is used to deliver stent 212, post dilate stent 212, and recover protective element 112 in accordance with one embodiment, the entire procedure is relatively simple, quick, and has a minimal associated risk of infection.

More particularly, FIG. 1 is a side view, in partial cross-section, of a protective device 102 deployed within a parent artery or vessel 104 of a patient adjacent to an occlusion 106 in accordance with one embodiment of the present invention. Occlusion 106 occludes or completely blocks blood flow through vessel 104. Illustratively, occlusion 106 is plaque, thrombi, other deposits, emboli or other substances on an inner vessel wall 108 of vessel 104. Occlusion 106 reduces the blood carrying capacity of vessel 104. Left untreated, occlusion 106 could cause serious and permanent injury, or even death to the patient.

Blood flow through vessel 104 is in the direction indicated by arrow 110. Protective device 102 is introduced intra-vascularly and guided through vessel 104 to occlusion 106 using any one of a number of techniques well known to those of skill in the art.

As shown in FIG. 1, protective device 102 includes a protective element 112, for example, a filter or protective balloon, at a distal end 114 of protective device 102. Protective device 102 further includes a guide wire 116 extending proximally from protective element 112.

Protective device 102 is guided through vessel 104 such that protective element 112 is located downstream from occlusion 106, i.e., in the direction of blood flow from occlusion 106. Protective element 112 is then anchored within vessel 104 using any one of a number of techniques and the particular technique used is not essential to the present invention, but are well known to persons skilled in the art.

In one embodiment, protective element 112 is permeable to blood, e.g., is a filter, such that blood flow continues through vessel 104 even after protective element 112 is anchored against inner vessel wall 108. Protective element 112 filters and captures any particulates contained within the blood flowing through protective element 112 in accordance with this embodiment.

In another embodiment, protective element 112 may be a protective balloon that completely occludes vessel 104 after protective element 112 is anchored against inner vessel wall 108. By preventing blood flow through vessel 104, a so configured protective element 112 would prevent any particulates from passing protective element 112. In this configuration, guide wire 116 includes a protective balloon inflation lumen (not shown).

FIG. 2 is a side view, in partial cross-section, of a stent delivery, dilation and filter recovery device 202 within vessel 104 configured in accordance with one embodiment according to the present invention. Referring now to FIGS. 1 and 2 together, stent delivery, dilation and filter recovery device 202 is advanced over guide wire 116 of protective device 102 to be adjacent to protective element 112.

More particularly, stent delivery, dilation and filter recovery device 202, sometimes called device 202, includes a tip 204 mounted on an inner member 205 that defines a guide wire lumen 206. In accordance with this embodiment, tip 204 is tapered, sometimes called olive-shaped, to facilitate advancing of device 202 over guide wire 116. More particularly, guide wire 116 is threaded through tip 204 and through guide wire lumen 206.

Mounted over inner member 205 is a stent dilation balloon 208 of device 202. Stent dilation balloon 208 is coupled to an outer shaft 209 of device 202 that defines a balloon dilation lumen 210. More particularly, balloon dilation lumen 210 is defined by the annular space between inner member 205 and outer shaft 209.

Stent dilation balloon 208 is communicatively coupled to balloon dilation lumen 210 of device 202. As discussed further below, stent dilation balloon 208 is inflated by fluid, which passes through balloon dilation lumen 210.

Mounted over stent dilation balloon 208 is a stent 212 of device 202. In accordance with one embodiment, stent dilation balloon 208 is ⅝ to ¾ the length of stent 212 but has other dimensions in other embodiments (not shown), e.g., is greater in length than stent 212.

Stent 212 is constrained within a sheath 214 of device 202. In accordance with this embodiment, stent 212 is a self expanding stent. Device 202 is positioned within vessel 104, e.g., using radiopaque markers (not shown), so that stent 212 is located within occlusion 106.

FIG. 3 is a side view, in partial cross-section, of stent delivery, dilation and filter recovery device 202 of FIG. 2 within vessel 104 after deployment of stent 212 in accordance with one embodiment of the present invention. Referring now to FIGS. 2 and 3 together, sheath 214 is retracted to expose stent 212. As stent 212 is exposed, stent 212 self expands into occlusion 106 thus providing a force opening that part of vessel 104. Outer shaft 209 includes a lip or other holding member which prevents movement of stent 212 during retraction of sheath 214. Outer shaft 209 and inner member 205 have a sufficient column strength to prevent buckling of outer shaft 209 and/or inner member 205 during retraction of sheath 214 and deployment of stent 212.

FIG. 4 is a side view, in partial cross-section, of the post deployment dilation of stent 212 in accordance with one embodiment of the present invention. Referring now to FIG. 4, stent dilation balloon 208 is inflated by fluid, which passes through balloon dilation lumen 210. Inflation of stent dilation balloon 208 causes stent dilation balloon 208 to press outwards on stent 212. This, in turn, causes stent 212 to further expand thus further opening vessel 104. In one embodiment, stent dilation balloon 208 is inflated one or more times, e.g., is inflated twice, to expand stent 212.

In another embodiment, a stent such as the one labelled 212 is not a self expanding stent. In accordance with this embodiment, referring again to FIG. 3, such a stent does not self expand upon retraction of sheath 214. However, inflation of stent dilation balloon 208 causes the stent to expand and to become anchored to inner vessel wall 108.

Although device 202 is illustrated and discussed above as including stent dilation balloon 208, in another embodiment, device 202 does not include stent dilation balloon 208. In accordance with this embodiment, stent 212 is self expanding and self expands upon retraction of sheath 214 as discussed above in reference to FIG. 3 to become anchored to inner vessel wall 108.

After deployment of stent 212, stent 212 is anchored to inner vessel wall 108. Stent 212 compresses occlusion 106 thus providing a larger pathway through vessel 104. Stated another way, stent 212 opens vessel 104.

Referring now to FIG. 4, as a result of the above procedure, particulates 412, e.g., debris and pieces from occlusion 106, are sometimes introduced into vessel 104. However, protective device 112, e.g., a filter, captures particulates 412 thus preventing particulates 412 from escaping into the vasculature of the patient and creating the associated complications.

FIG. 5 is a side view, in partial cross-section, of the enclosure of stent dilation balloon 208 and tip 204 within sheath 214 in accordance with one embodiment of the present invention. Referring now to FIGS. 4 and 5 together, stent dilation balloon 208 is deflated. Sheath 214 is advanced distally and towards protective element 112.

More particularly, sheath 214 is advanced over stent dilation balloon 208 and through stent 212. Sheath 214 has an outer diameter OD less than an inner diameter ID of stent 212 after deployment, i.e., after stent 212 is expanded. This allows sheath 214 to be moved forward into and through stent 212 and over stent dilation balloon 208.

In accordance with one embodiment, as sheath 214 is advanced over stent dilation balloon 208, sheath 214 assists in collapsing stent dilation balloon 208 and encloses, sometimes called encapsulates, stent dilation balloon 208 within sheath 214. Stent dilation balloon 208 is secured (held stationary), e.g., by securing inner member 205 or outer shaft 209, and thus prevented from movement during movement of sheath 214.

In addition, sheath 214 is advanced distally and towards protective element 112 and over (past) tip 204. As sheath 214 is advanced over tip 204, sheath 214 collapses (compresses) tip 204 to enclose tip 204 within sheath 214. In one embodiment, tip 204 is an elastic member thus allowing tip 204 to be collapsed (compressed) by sheath 214. Tip 204 is secured (held stationary), e.g., by securing inner member 205, and thus prevented from movement during movement of sheath 214.

FIG. 6 is a side view, in partial cross-section, of the enclosure of protective element 112 within sheath 214 in one embodiment according to the present invention. Referring now to FIGS. 5 and 6 together, sheath 214 is further advanced distally and over protective element 112. More particularly, sheath 214 is advanced distally, e.g., in a first direction, past tip 204 and over protective element 112. As sheath 214 is advanced over protective element 112, sheath 214 collapses (or compresses) protective element 112 and encloses protective element 112 within sheath 214. Protective element 112 is secured (held stationary), e.g., by tightening a torque handle onto guide wire 116, and thus prevented from movement during movement of sheath 214.

Device 202 including protective device 102 are then retracted, i.e., moved in the proximal direction. Since stent dilation balloon 208, tip 204, and protective element 112 are enclosed within sheath 214, any possibility of stent dilation balloon 208, tip 204, and/or protective element 112 catching on stent 212 during retraction is eliminated. In this manner, dislocation of stent 212 and the associated complications are avoided.

Further, since only device 202 is used to deliver stent 212, post dilate stent 212, and recover protective element 112, the entire procedure is relatively simple, quick, and has a minimal associated risk of infection.

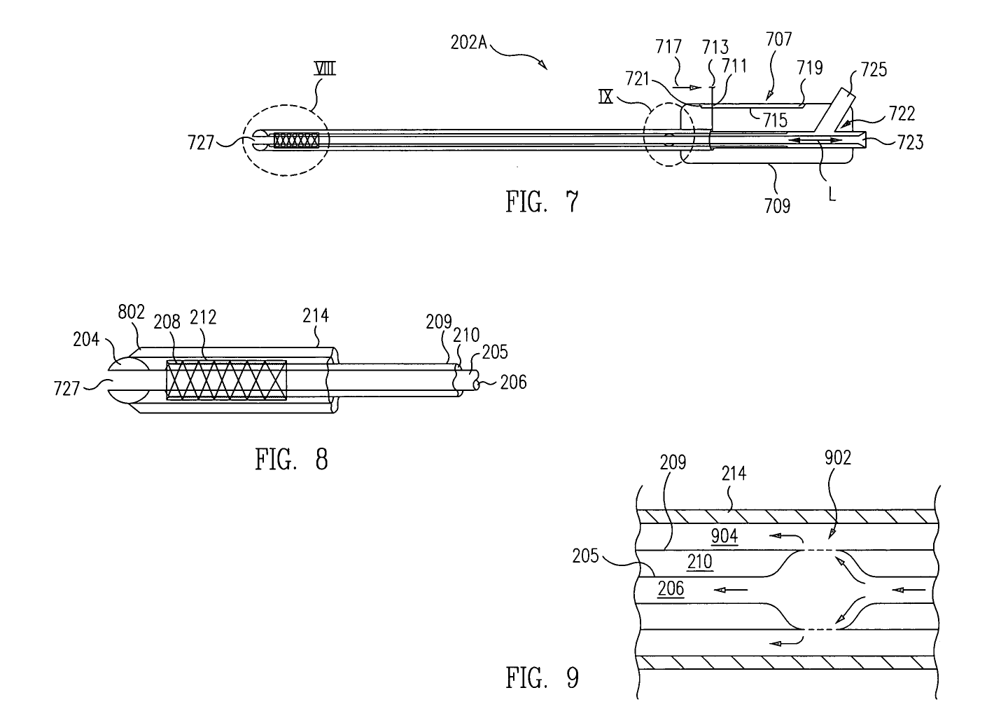

FIG. 7 is a side view, in partial cross-section, of a stent delivery, dilation and filter recovery device 202A, sometimes called device 202A, in one embodiment according to the present invention. FIG. 8 is enlarged view of the region VIII of FIG. 7. Referring now to FIGS. 7 and 8 together, device 202A includes tip 204, inner member 205 that defines guide wire lumen 206, stent dilation balloon 208, outer shaft 209 that defines balloon dilation lumen 210, stent 212, and sheath 214 as discussed above.

As illustrated in FIG. 8, sheath 214 includes a tapered end 802 at the distal end of sheath 214. Tapered end 802 facilitates advancement of device 202A through the vasculature of the patient.

Device 202A further includes a handle 707. Handle 707 includes a housing 709 and a sheath movement mechanism 711 slidably coupled to housing 709. Sheath movement mechanism 711 is coupled to sheath 214. Further, sheath movement mechanism 711 includes an actuation button 713. Sheath movement mechanism 711 extends from inside housing 709 through a sheath movement mechanism slot 715 of housing 709.

In accordance with this embodiment, depression of actuation button 713 (or its circumferential rotation) disengages sheath movement mechanism 711 from housing 709. This allows sheath movement mechanism 711, and thus sheath 214, to be moved longitudinally along a longitudinal axis L of a handle 707.

More particularly, depression of actuation button 713 allows sheath movement mechanism 711 to be moved back and forth, sometimes called proximally and distally, within sheath movement mechanism slot 715. This, in turn, moves sheath 214 back and forth.

To allow sheath movement mechanism 711 to be moved back and forth within sheath movement mechanism slot 715, sheath movement mechanism 711 is located between a proximal, e.g., first, end 719 and a distal, e.g., second, end 721 of sheath movement mechanism slot 715. Stated another way, sheath movement mechanism 711 is not butted up against either proximal end 719 or distal end 721 of sheath movement mechanism slot 715 prior to deployment of stent 212.

Illustratively, sheath movement mechanism 711 is slid proximally in the direction of arrow 717 to retract sheath 214 and deploy stent 212 as discussed above in reference to FIG. 3. Further, sheath movement mechanism 711 is slid distally in the direction opposite to arrow 717 to advance sheath 214 though stent 212 after deployment and to enclose stent dilation balloon 208, tip 204 and protective element 112 as discussed above in reference to FIGS. 5 and 6.

Handle 707 further includes a Y adapter 722 having a guide wire port 723 and a balloon inflation port 725. Guide wire port 723 is communicatively coupled with guide wire lumen 206. During use, guide wire 116 (FIG. 1) is threaded through a distal end 727 of guide wire lumen 206 and exits guide wire port 723 of handle 707.

Balloon inflation port 725 is communicatively coupled with balloon dilation lumen 210 and thus stent dilation balloon 208. During use, a fluid, e.g., saline solution, is injected into balloon inflation port 725 to inflate stent dilation balloon 208 and discussed above in reference to FIG. 4. Further, this fluid is withdrawn from balloon inflation port 725 to deflate stent dilation balloon 208.

FIG. 9 is enlarged view of the region IX of FIG. 7. Referring now to FIGS. 7 and 9 together, a perfusion joint 902 is between a sheath lumen 904 and guide wire lumen 206. More particularly, sheath lumen 904 is defined by the annular space between outer shaft 209 and sheath 214.

Perfusion joint 902 communicatively couples sheath lumen 904 and guide wire lumen 206. More particularly, perfusion joint 902 allows fluid to pass directly between sheath lumen 904 and guide wire lumen 206 without introducing the fluid into balloon dilation lumen 210. Stated another way, balloon dilation lumen 210 is not communicatively coupled to either guide wire lumen 206 or sheath lumen 904.

In one embodiment according to the present invention, stent delivery, dilation and filter recovery device 202A is flushed to remove any air prior to being introduced into the vasculature of the patient. In accordance with this embodiment, a flushing solution, e.g., a saline solution, is injected through guide wire port 723. The flushing solution flows through and fills guide wire lumen 206 thus removing any air from guide wire lumen 206.

After a sufficient flow of the flushing solution has passed through guide wire lumen 206 to ensure that all air has been removed from guide wire lumen 206, distal end 727 of guide wire lumen 206 is plugged, e.g., with a finger of the physician. The flushing solution injected through guide wire port 723 is thus forced through perfusion joint 902 and into sheath lumen 904. The flushing solution flows through and fills sheath lumen 904 thus removing any air from sheath lumen 904.

This disclosure provides exemplary embodiments according to the present invention. The scope of the present invention is not limited by these exemplary embodiments. Numerous variations, whether explicitly provided for by the specification or implied by the specification or not, such as variations in structure, dimension, type of material and manufacturing process may be implemented by one of skill in the art in view of this disclosure.

What is claimed is:

1. A device for delivering a stent comprising:
a sheath;
an inner member defining a guide wire lumen;
a tip mounted on said inner member; and
a handle comprising a sheath movement mechanism coupled to said sheath, said sheath movement mechanism for advancing said sheath over said tip.

2. The device of claim 1 further comprising a stent dilation balloon mounted over said inner member.

3. The device of claim 2 further comprising an outer shaft coupled to said stent dilation balloon.

4. The device of claim 3 wherein a balloon dilation lumen is defined by an annular space between said inner member and said outer shaft.

5. The device of claim 4 wherein said stent dilation balloon is communicatively coupled to said balloon dilation lumen.

6. The device of claim 5 wherein said handle comprises a balloon inflation port communicatively coupled with said balloon dilation lumen and said stent dilation balloon.

7. The device of claim 2 wherein said stent is mounted over said stent dilation balloon.

8. The device of claim 7 wherein said stent is a self expanding stent.

9. The device of claim 8 wherein retraction of said sheath deploys said stent.

10. The device of claim 9 wherein said sheath has an outer diameter less than an inner diameter of said sheath after deployment.

11. The device of claim 10 wherein said sheath movement mechanism is further for advancing said sheath through said stent after deployment.

12. The device of claim 1 wherein said tip is tapered.

13. The device of claim 1 wherein said sheath comprises a tapered end.

14. The device of claim 1 wherein said handle comprises a guide wire port communicatively coupled to said guide wire lumen.

15. The device of claim 1 wherein a sheath lumen is defined by an annular space between an outer shaft and said sheath, said device further comprising a perfusion joint between said sheath lumen and said guide wire lumen.

16. A device for delivering a stent comprising:
a sheath; and
a means for moving said sheath coupled to said sheath, said means for moving for retracting said sheath to deploy said stent and for advancing said sheath to encapsulate a protective element used with said device.

17. The device of claim 16 further comprising a means for expanding said stent after deployment.

18. A device for delivering a stent comprising:
an inner member defining a guide wire lumen;
a tip mounted on said inner member;
a stent dilation balloon mounted over said inner member, wherein said stent is mounted over said stent dilation balloon;
an outer shaft coupled to said stent dilation balloon, wherein a balloon dilation lumen is defined by an annular space between said inner member and said outer shaft;
a sheath constraining said stent; and
a sheath movement mechanism for advancing said sheath past said tip.

19. The device of claim 18 wherein a handle comprises said sheath movement mechanism and a sheath movement mechanism slot, said sheath movement mechanism being between a first end and a second end of said sheath movement mechanism slot.

20. The device of claim 19 wherein said handle comprises:
a guide wire port communicatively coupled to said guide wire lumen; and
a balloon inflation port communicatively coupled to said balloon dilation lumen.

21. A method comprising:
deploying a protective device comprising a protective element adjacent an occlusion in a vessel;
advancing a device comprising a stent and a sheath over a guide wire of said protective device;
retracting said sheath to deploy said stent; and
advancing said sheath through said stent and over said protective element.

22. The method of claim 21 wherein said advancing said sheath further comprises collapsing said protective element with said sheath.

23. The method of claim 21 further comprising retracting said protective element through said stent, said protective element being enclosed within said sheath during said retracting.

24. The method of claim 23 wherein said sheath eliminates any possibility of said protective element catching on said stent.

25. The method of claim 21 wherein said protective element is a filter.

26. The method of claim 21 further comprising inflating a stent dilation balloon to expand said stent.

27. The method of claim 26 further comprising deflating said stent dilation balloon, wherein said advancing said sheath further comprises advancing said sheath over said stent dilation balloon after said deflating.

28. The method of claim 27 wherein said advancing said sheath further comprises collapsing said stent dilation balloon with said sheath.

29. The method of claim 21 wherein said advancing said sheath further comprises advancing said sheath over a tip mounted on an inner member of said device through which said guide wire extends.

30. The method of claim 29 wherein said advancing said sheath further comprises collapsing said tip with said sheath.
